(19) United States
(12) Patent Application Publication
(10) Pub. No.: US 2026/0091151 A1
(43) Pub. Date: Apr. 2, 2026

(54) Aerosolized Disinfectant Device

(71) Applicant: Micheal Stevenson, North Las Vegas, NV (US)

(72) Inventor: Micheal Stevenson, North Las Vegas, NV (US)

(21) Appl. No.: 18/904,752

(22) Filed: Oct. 2, 2024

(51) Int. Cl. A61L 2/22; A61L 2/24; A61L 2/26

(52) U.S. Cl. CPC A61L 2/22; A61L 2/24; A61L 2/26; A61L 2103/75; A61L 2202/14; A61L 2202/15; A61L 2202/16

(57) ABSTRACT

An aerosolized disinfectant device for dispensing a disinfectant fluid in an unoccupied room includes a housing having an interior space. A cannister is positioned within the interior space and holds an aerosolized disinfectant fluid. A cap is pivotably coupled to the housing. A motor and a pump are electrically coupled to a processor. The motor is actuatable to pivot the cap. The pump is fluidly coupled to the cannister to receive and pressurize the aerosolized disinfectant fluid. A plurality of cap nozzles fluidly coupled to the pump dispense the aerosolized disinfectant fluid while the cap is being pivoted. A timer with a timer setting is electrically coupled to the processor. The processor actuates the pump and the motor once the timer setting has expired.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to disinfectant sprays and more particularly pertains to a new disinfectant spray for dispensing a disinfectant fluid in an unoccupied room.

### (2) Description of Related Art Including Information Disclosed Under 37 CFR 1.97 and 1.98

The prior art relates to disinfectant sprays. Disinfectant sprays are a popular tool for controlling the spread of germs and inhibiting the growth of microorganisms. During the COVID-19 public health crisis, disinfectant solutions and sprays became exponentially more popular. For example, aerosolized disinfectant sprays allow users to quickly and efficiently disinfect surfaces. However, the user must physically hold the spray canister to apply the spray to each surface. Because of the close proximity required between the user and the aerosol can, users may breathe in the spray, or may be exposed to the germs and bacteria on the unsensitized surfaces. Thus, there is a need in the art for a disinfectant dispensing system that will release disinfectant solutions onto surfaces in uninhabited rooms.

### BRIEF SUMMARY OF THE INVENTION

An embodiment of the disclosure meets the needs presented above by generally comprising a housing having an interior space. A cannister is positioned within the interior space of the housing. The cannister is configured to hold an aerosolized disinfectant fluid. A cap is pivotably coupled to the housing wherein the cap is pivotable between a first lateral position and a second lateral position. A power source is positioned within the interior space of the housing. A processor is electrically coupled to the power source.

A motor is electrically coupled to the processor. The motor is operably coupled to the cap wherein the motor is actuatable to pivot the cap between the first lateral position and the second lateral position. A pump is electrically coupled to the processor. The pump is fluidly coupled to the cannister wherein the pump is configured to receive and to pressurize the aerosolized disinfectant fluid. A plurality of cap nozzles is fluidly coupled to the pump. The plurality of cap nozzles is exposed within an exterior surface of the cap wherein the plurality of cap nozzles are configured to dispense the aerosolized disinfectant fluid outwardly from the housing while the cap is being pivoted between the first lateral position and the second lateral position.

A timer is electrically coupled to the processor. The timer includes a display panel that is positioned on the exterior surface of the cap. The display panel is configured to display a timer setting of the timer. The processor is configured to actuate the pump and the motor once the timer setting has expired. A control panel is actuatable to set the timer setting.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top isometric view of an aerosolized disinfectant device according to an embodiment of the disclosure.

FIG. 2 is a bottom isometric view of an embodiment of the disclosure.

FIG. 3 is a top view of an embodiment of the disclosure.

FIG. 4 is a cross-sectional view of an embodiment of the disclosure.

FIG. 5 is a cross-sectional view of an embodiment of the disclosure.

FIG. 6 is an in-use view of an embodiment of the disclosure.

FIG. 7 is a block diagram view of an embodiment of the disclosure.

### DETAILED DESCRIPTION OF THE INVENTION

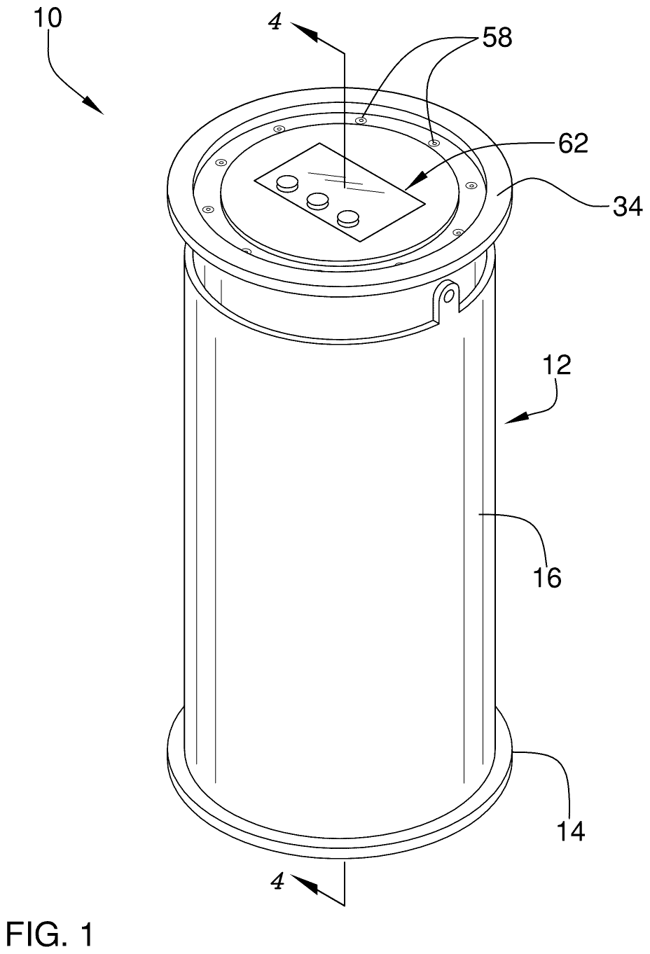

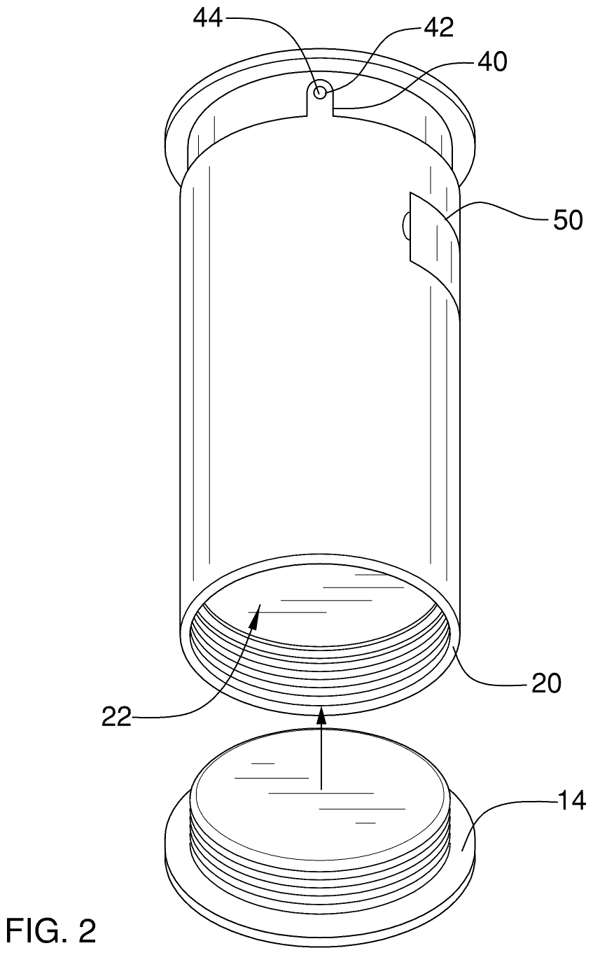

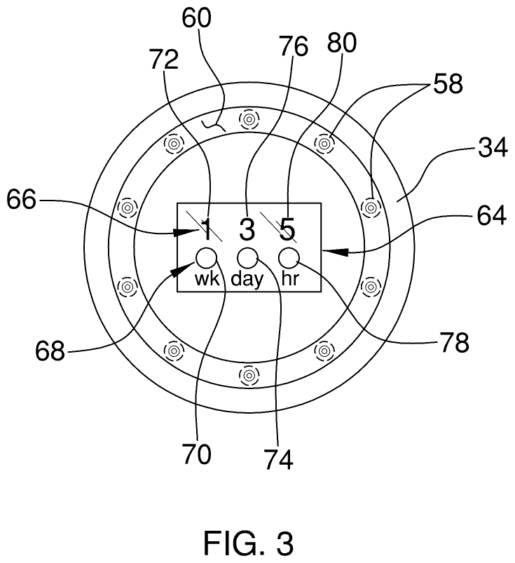

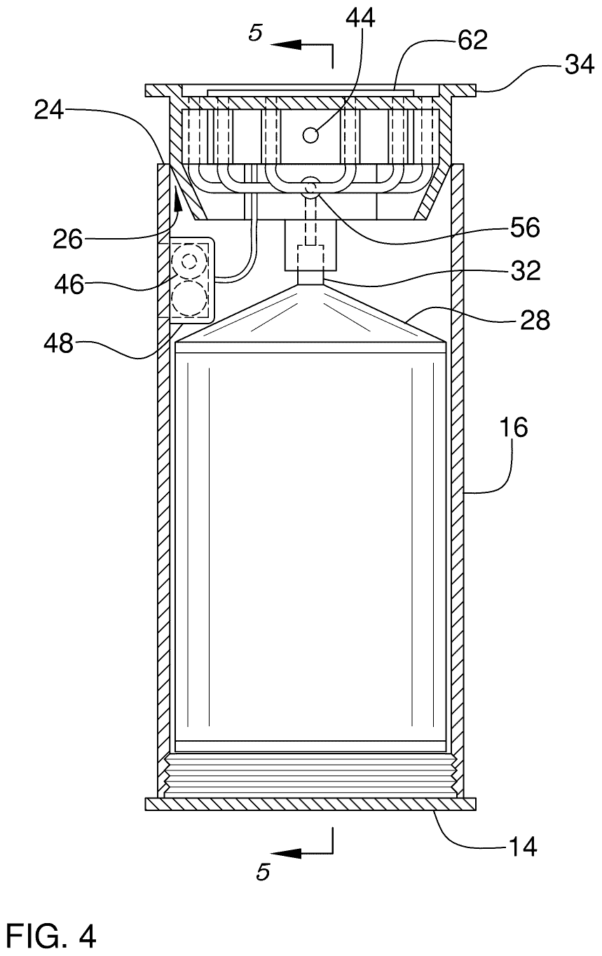

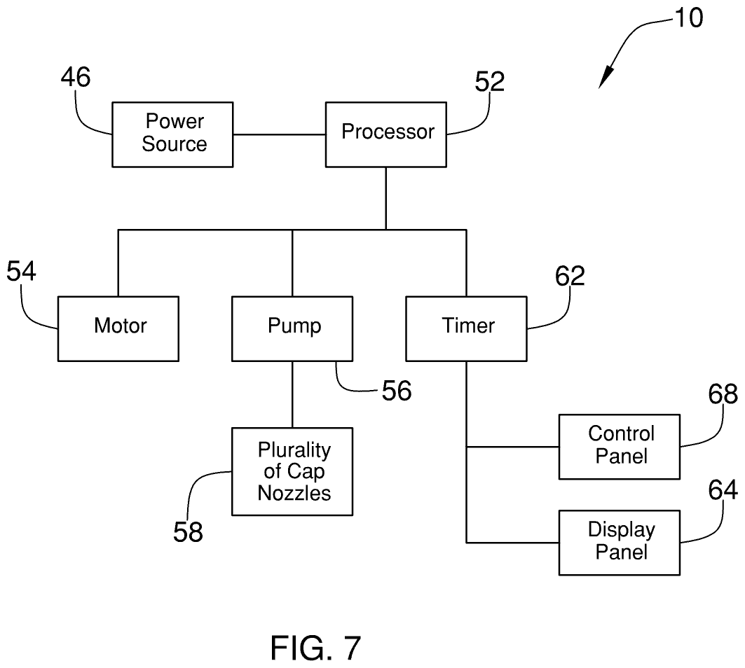

With reference now to the drawings, and in particular to FIGS. 1 through 7 thereof, a new disinfectant spray embodying the principles and concepts of an embodiment of the disclosure and generally designated by the reference numeral 10 will be described.

As best illustrated in FIGS. 1 through 7, the aerosolized disinfectant device 10 generally comprises a housing 12, which generally includes an interior space 18. For example, the housing 12 may have a base 14 and a peripheral wall 16 that is coupled to and extends upwardly from the base 14 to define the interior space 18. The base 14 may be threadably coupled to the peripheral wall 16 wherein the base 14 is removable from the peripheral wall 16 to expose the interior space 18. The housing 12 may be cylindrical as shown in the Figures, although other shapes, such as rectangular prisms, are also contemplated.

The peripheral wall 16 may have a lower edge 20 that is positionable against the base 14 when the base 14 is coupled to the peripheral wall 16. The lower edge 20 may define a lower opening 22 into the interior space 18. The peripheral wall 16 may have an upper edge 24 that is distally positioned relative to the lower edge 20. The upper edge 24 may define an upper opening 26 into the interior space 18.

A cannister 28 is positioned within the interior space 18 of the housing 12. The cannister 28 is configured to hold an aerosolized disinfectant fluid 30. The aerosolized disinfectant fluid 30 is generally an antibacterial fluid wherein the aerosolized disinfectant fluid 30 is configured to inhibit viruses, germs, bacteria, and other microorganisms from being collected on a surface on which the aerosolized disinfectant fluid 30 is dispensed. The aerosolized disinfectant fluid 30 may also kill or remove such microorganisms from the surface. In some embodiments, the cannister 28 may be removably positionable within the interior space 18. For example, the lower opening 22 may have a size exceeding a size of the cannister 28 wherein the cannister 28 is positionable within the interior space 18 through the lower opening 22.

A cannister nozzle 32 may be coupled to the cannister 28. The cannister nozzle 32 is configured to dispense the aerosolized disinfectant fluid 30 from the cannister 28.

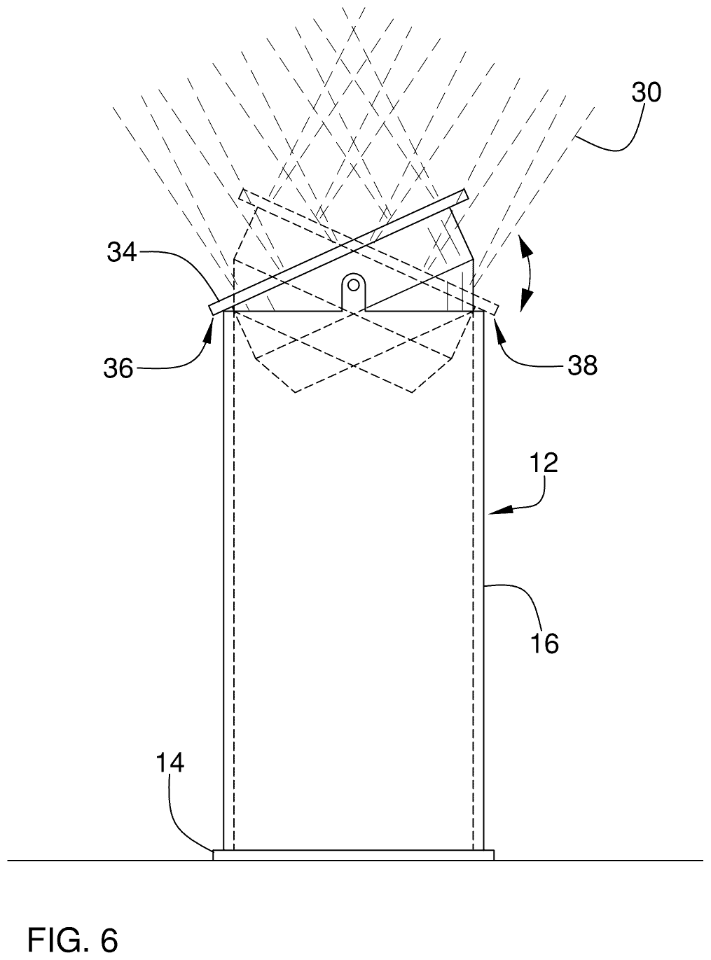

A cap 34 is pivotably coupled to the housing 12. As shown in the figures, the cap 34 may be positioned proximate to the upper edge 24 of the peripheral wall. The cap 34 is generally pivotable within the upper opening 26 between a first lateral position 36 and a second lateral position 38, as shown in FIG. 6.

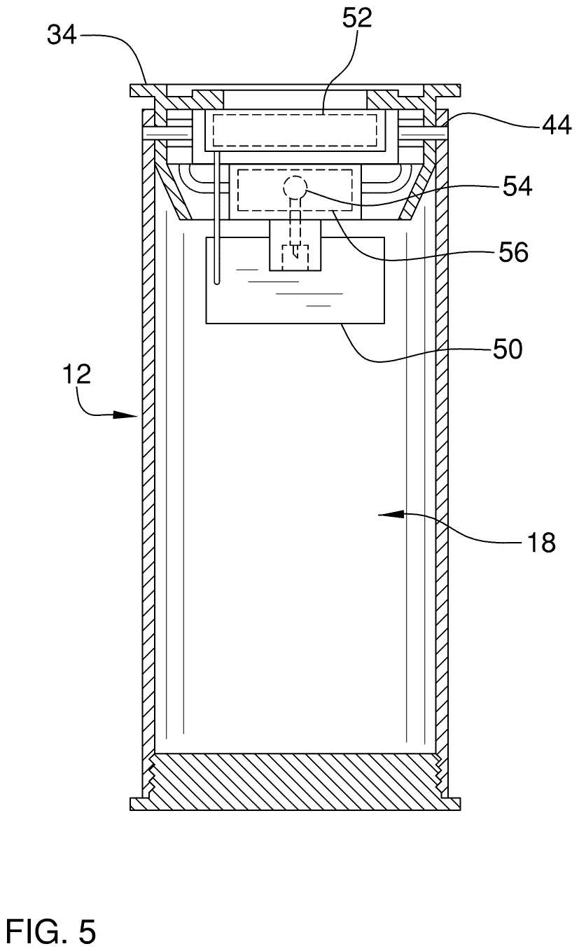

A pair of projections 40 may be coupled to and extend upwardly from the upper edge 24 of the peripheral wall 16. The pair of projections 40 may be aligned with each other along a diameter of the upper opening 26, for example when the housing 12 is cylindrical as shown in FIG. 5. In other embodiments, such as when the housing 12 is a rectangular prism, the pair of projections 40 may be aligned with each other across the upper opening 26. The pair of projections 40 are generally positioned on opposing sides of the peripheral wall 16.

The aerosolized disinfectant device 10 may further include a pair of apertures 42. Each aperture of the pair of apertures 42 generally extends through a respective projection of the pair of projections 40. A pin 44 may extend through the pair of apertures 42. The pin 44 may be coupled to the cap 34 wherein the cap 34 is pivotable about the pin 44 between the first lateral position 36 and the second lateral position 38. As shown in FIG. 5, the pin 44 may comprise a single piece that extends through both apertures of the pair of apertures 42. In other embodiments, the pin 44 may comprise a pair of pins 44. Each pin 44 of the pair of pins 44 may extend through a respective aperture of the pair of apertures 42.

A power source 46 may be positioned within the interior space 18 of the housing 12. For example, the power source 46 may comprise a battery.

A compartment 48 may be coupled to the peripheral wall 16 of the housing 12. For example, the compartment 48 may extend into the interior space 18 of the housing 12. The compartment 48 generally has a size exceeding a size of the power source 46 wherein the power source 46 is positionable within the compartment 48.

An access door 50 may be coupled to the peripheral wall 16 of the housing 12. The access door 50 is generally openable to selectively expose the compartment 48 wherein the access door 50 is configured to facilitate removal of the power source 46 from the housing 12. For example, the access door 50 may be opened to recharge or replace the battery.

A processor 52 is electrically coupled to the power source 46. The processor 52 may be positioned within the cap 34, as shown in FIG. 5.

A motor 54 is electrically coupled to the processor 52. The motor 54 is operably coupled to the cap 34 wherein the motor 54 is actuatable to pivot the cap 34 between the first lateral position 36 and the second lateral position 38.

A pump 56 is electrically coupled to the processor 52. The pump 56 is fluidly coupled to the cannister 28 wherein the pump 56 is configured to receive and to pressurize the aerosolized disinfectant fluid 30. For example, the pump 56 may be fluidly coupled to the cannister nozzle 32. The pump 56 may be positioned within the cap 34.

A plurality of cap nozzles 58 is fluidly coupled to the pump 56. The plurality of cap nozzles 58 is generally exposed within an exterior surface 60 of the cap 34. The plurality of cap nozzles 58 is configured to dispense the aerosolized disinfectant fluid 30 outwardly from the housing 12 while the cap 34 is being pivoted between the first lateral position 36 and the second lateral position 38.

A timer 62 is electrically coupled to the processor 52. The timer 62 generally includes a display panel 64. The display panel 64 may be positioned on the exterior surface 60 of the cap 34. The display panel 64 generally displays a timer setting 66 of the timer 62. The processor 52 actuates the pump 56 and the motor 54 once the timer setting 66 has expired.

A control panel 68 is actuatable to set the timer setting 66. For example, the control panel 68 may include a week button 70 that is actuatable to adjust a week number 72 of the timer setting 66. For example, the week button 70 may be actuated to increase or decrease a number of weeks that the user wants the timer 62 to track. A day button 74 may be actuatable to adjust a day number 76 of the timer setting 66. For example, the day button 74 may be actuated to increase or decrease a number of days that the user wants the timer 62 to track. An hour button 78 may be actuatable to adjust an hour number 80 of the timer setting 66. For example, the hour button 78 may be actuated to increase or decrease a number of hours that the user wants the timer 62 to track.

In use, the housing 12 can be placed on a surface, such as the floor or a tabletop, within a room that the user wants to disinfect. The timer setting 66 of the timer 62 can be set using the control panel 68, and the user and all other individuals can vacate the room. Upon expiration of the timer setting 66, the processor 52 may actuate the pump 56 and the motor 54. The motor 54 will pivot the cap 34 between the first lateral position 36 and the second lateral position 38 while the aerosolized disinfectant fluid 30 is expelled from the cannister 28 through the plurality of cap nozzles 58. Thus, the room can be disinfected while the room is vacant.

In embodiments where the base 14 is threadably coupled to the peripheral wall 16, the base 14 can be removed to expose the lower opening 22 and remove the cannister 28, for example when the canister is emptied. The cannister 28 can be refilled, or replaced, and repositioned in the interior space 18 through the lower opening 22. The housing 12 may thus be reused. In embodiments where the base 14 is fixedly coupled to the peripheral wall 16, the housing 12 may be discarded once the cannister 28 is emptied.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An aerosol spray assembly comprising:
a housing having an interior space;
a cannister being positioned within the interior space of the housing, the cannister being configured to hold an aerosolized disinfectant fluid;
a cap being pivotably coupled to the housing wherein the cap is pivotable between a first lateral position and a second lateral position;
a power source being positioned within the interior space of the housing;
a processor being electrically coupled to the power source;
a motor being electrically coupled to the processor, the motor being operably coupled to the cap wherein the motor is actuatable to pivot the cap between the first lateral position and the second lateral position;
a pump being electrically coupled to the processor, the pump being fluidly coupled to the cannister wherein the pump is configured to receive and to pressurize the aerosolized disinfectant fluid;
a plurality of cap nozzles being fluidly coupled to the pump, the plurality of cap nozzles being exposed within an exterior surface of the cap wherein the plurality of cap nozzles are configured to dispense the aerosolized disinfectant fluid outwardly from the housing while the cap is being pivoted between the first lateral position and the second lateral position; and
a timer being electrically coupled to the processor, the timer including:
a display panel displaying a timer setting of the timer, the processor actuating the pump and the motor once the timer setting has expired; and
a control panel being actuatable to set the timer setting.

2. The aerosol spray assembly of claim 1, further comprising a compartment being coupled to the peripheral wall of the housing, the compartment extending into the interior space of the housing, the compartment having a size exceeding a size of the power source wherein the power source is positionable within the compartment.

3. The aerosol spray assembly of claim 2, further comprising an access door being coupled to the peripheral wall of the housing, the access door being openable to selectively expose the compartment wherein the access door is configured to facilitate removal of the power source from the housing.

4. The aerosol spray assembly of claim 1, the control panel further comprising an hour button being actuatable to adjust an hour number of the timer.

5. The aerosol spray assembly of claim 4, the control panel further comprising a day button being actuatable to adjust a day number of the timer setting.

6. The aerosol spray assembly of claim 5, the control panel further comprising a week button being actuatable to adjust a week number of the timer setting.

7. The aerosol spray assembly of claim 1, the housing further comprising a base and a peripheral wall being coupled to and extending upwardly from the base to define the interior space.

8. The aerosol spray assembly of claim 7, wherein the base is threadably coupled to the peripheral wall wherein the base is removable from the peripheral wall to expose the interior space, the cannister being removably positionable within the interior space when the base is removed from the peripheral wall.

9. The aerosol spray assembly of claim 7, the peripheral wall further comprising an upper edge being distally positioned relative to the base, the upper edge defining an upper opening into the interior space, the cap being pivotable within the upper opening between the first lateral position and the second lateral position.

10. An aerosol spray assembly comprising:
a housing having a base and a peripheral wall being coupled to and extending upwardly from the base to define an interior space, the base being threadably coupled to the peripheral wall wherein the base is removable from the peripheral wall to expose the interior space, the peripheral wall having a lower edge being positionable against the base when the base is coupled to the peripheral wall, the lower edge defining a lower opening into the interior space, the peripheral wall having an upper edge being distally positioned relative to the lower edge, the upper edge defining an upper opening into the interior space;
a cannister being removably positionable within the interior space of the housing, the cannister being configured to hold an aerosolized disinfectant fluid;
a cannister nozzle being coupled to the cannister wherein the cannister nozzle is configured to dispense the aerosolized disinfectant fluid from the cannister;
a cap being pivotably coupled to the peripheral wall of the housing wherein the cap is pivotable within the upper opening between a first lateral position and a second lateral position;
a power source being positioned within the interior space of the housing, the power source comprising a battery;
a compartment being coupled to the peripheral wall of the housing, the compartment extending into the interior space of the housing, the compartment having a size exceeding a size of the power source wherein the power source is positionable within the compartment;
an access door being coupled to the peripheral wall of the housing, the access door being openable to selectively expose the compartment wherein the access door is configured to facilitate removal of the power source from the housing;
a processor being positioned within the cap, the processor being electrically coupled to the power source;
a motor being electrically coupled to the processor, the motor being operably coupled to the cap wherein the motor is actuatable to pivot the cap between the first lateral position and the second lateral position;
a pump being electrically coupled to the processor, the pump being fluidly coupled to the cannister nozzle of the cannister wherein the pump is configured to receive and to pressurize the aerosolized disinfectant fluid, the pump being positioned within the cap;
a plurality of cap nozzles being fluidly coupled to the pump, the plurality of cap nozzles being exposed within an exterior surface of the cap wherein the plurality of cap nozzles are configured to dispense the aerosolized disinfectant fluid outwardly from the housing while the cap is being pivoted between the first lateral position and the second lateral position; and
a timer being electrically coupled to the processor, the timer including:
a display panel being positioned on the exterior surface of the cap, the display panel being configured to display a timer setting of the timer, the processor being configured to actuate the pump and the motor once the timer setting has expired; and
a control panel being actuatable to set the timer setting, the control panel including:
a week button being actuatable to adjust a week number of the timer setting;
a day button being actuatable to adjust a day number of the timer setting; and
an hour button being actuatable to adjust an hour number of the timer setting.
